(19) United States
(12) Patent Application Publication
(10) Pub. No.: US 2004/0247550 A1
(43) Pub. Date: Dec. 9, 2004

(54) Hair or skin conditioning composition comprising hydrophobically modified cationic thickening polymer

(75) Inventors: Satomi Asari, Kobe (JP); Susan Marie Guskey, Kobe (JP); Golam Faruque Khan, Kobe (JP); Eric Scott Johnson, Hamilton, OH (US); Robert Lee Wells, Kobe (JP)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/861,730

(22) Filed: Jun. 4, 2004

Related U.S. Application Data
(60) Provisional application No. 60/476,707, filed on Jun. 6, 2003.

(51) Int. Cl. A61K 7/06; A61K 7/11

(52) U.S. Cl. 424/70.12

(57) ABSTRACT

Disclosed is a hair or skin conditioning composition comprising by weight: (a) from about 0.01% to about 5% of a hydrophobically modified cationic thickening polymer; (b) from about 0.05% to about 10% of a surfactant selected from the group consisting of cationic surfactant, nonionic surfactant, and mixtures thereof; and (c) from about 0.1% to about 10% of a hydrophobically modified silicone; and (d) an aqueous carrier. The conditioning composition of the present invention has a suitable rheology for conditioning compositions, and provides conditioning benefits. The composition is especially suitable for hair care products such as hair conditioning products for rinse-off use.

### CROSS REFERENCE TO RELATED APPLICATION

This application claims the benefit of U.S. Provisional Application No. 60/476,707, filed on Jun. 6, 2003.

### FIELD OF THE INVENTION

The present invention relates to a conditioning composition comprising a hydrophobically modified cationic thickening polymer, a cationic or nonionic surfactant, and a hydrophobically modified silicone. The conditioning composition of the present invention has a suitable rheology for conditioning compositions and provides conditioning benefits. The composition is especially suitable for hair care products such as hair conditioning products for rinse-off use.

### BACKGROUND OF THE INVENTION

A variety of conditioning compositions such as hair conditioning compositions and skin conditioning compositions have been used for a variety of substances such as hair and skin. A common method of providing conditioning benefits is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds, are believed to provide a gel matrix which has a suitable rheology for conditioning compositions and which is suitable for providing a variety of conditioning benefits, especially when used for hair care products, such as slippery feel, softness and reduced tangling on wet hair and softness and moisturized feel on the hair when they are dried.

There exists a need for achieving the suitable rheology for conditioning compositions by other methods than forming the above gel matrix, while maintaining the conditioning benefits of the gel matrix.

Additionally, most of the above conditioning agents are also known to make the composition opaque. Thus, there is a need for conditioning compositions having a clear product appearance i.e., transparent or translucent product appearance.

Furthermore, most of the above conditioning agents are also known to weigh down the hair, when these conditioning agents are included in hair care compositions. For consumers who desire maintaining or increasing hair volume such as consumers having fine hair, the hair weighing down is not desirable. Thus, there is a need for hair conditioning compositions which do not weigh down the hair while providing conditioning benefits.

There also exists a need for conditioning compositions which consumers feel are easy to rinse-off while providing conditioning benefits, when the compositions are used in a form of rinse-off products. Conditioner compositions containing the above gel matrix also provide long-lasting slippery feel when rinsing the substance. Thus, there is a need for conditioning compositions which can easily leave the substance with a clean feel when rinsing the substance, while depositing sufficient amount of conditioning agents on the substance.

Based on the foregoing, there remains a need for conditioning compositions which have a suitable rheology for conditioning compositions by other methods than a gel matrix comprised by cationic surfactants and high melting point fatty compounds, while providing conditioning benefits, especially softness and reduced tangling on wet hair when used for hair care products such as hair conditioning products. There is also a need for such conditioning compositions which are suitable for providing further benefits such as, clear product appearance, not weighing down the hair, and easy to rinse-off feel, while providing the above rheological and conditioning benefits.

None of the existing art provides all of the advantages and benefits of the present invention.

### SUMMARY OF THE INVENTION

The present invention is directed to a hair or skin composition comprising by weight:

(a) from about 0.01% to about 5% of a hydrophobically modified cationic thickening polymer;

(b) from about 0.05% to about 10% of a surfactant selected from the group consisting of cationic surfactant, nonionic surfactant, and mixtures thereof;

(c) from about 0.1% to about 10% of a hydrophobically modified silicone; and

(d) an aqueous carrier.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Compositions

The hair or skin composition of the present invention comprises by weight:

(a) from about 0.01% to about 5% of a hydrophobically modified cationic thickening polymer;

(b) from about 0.05% to about 10% of a surfactant selected from the group consisting of cationic surfactant, nonionic surfactant, and mixtures thereof;

(c) from about 0.1% to about 10% of a hydrophobically modified silicone; and

(d) an aqueous carrier.

The conditioning compositions of the present invention have a suitable rheology for conditioning compositions and provide conditioning benefits, especially softness and reduced tangling on wet hair when used for hair care products such as hair conditioning products. The conditioning compositions of the present invention are suitable for providing further benefits such as clear product appearance, not weighing down the hair, and easy to rinse-off feel, while providing the above rheological and conditioning benefits. Thus, the composition of the present invention can provide clear product appearance in addition to the above rheological and conditioning benefits. When used for hair care products, the composition of the present invention provides the above rheological and conditioning benefits while not weighing down the hair. Furthermore, when used in a form of rinse-off products, the composition of the present invention can provide easy to rinse-off feel while providing the above rheological and conditioning benefits.

Without intending to be limited by theory, it is believed that the hydrophobically modified cationic thickening polymers interact with the surfactant and the hydrophobically modified silicone to form a physically crosslinked matrix which can provide a suitable rheology for conditioning compositions without the existence of a gel matrix comprised by cationic surfactants and high melting point fatty compounds, while providing conditioning benefits, especially softness and reduced tangling on wet hair when used for hair care products such as hair conditioning products.

Preferably, the composition of the present invention is substantially free of water-insoluble oily compound selected from hydrocarbons, fatty compounds, and mixtures thereof. In the present invention, the composition being “substantially free of water-insoluble oily compound” means that the composition includes 1.0% or less, preferably 0.5% or less, more preferably 0.1% or less, still more preferably 0% of water-insoluble oily compounds. The water-insoluble oily compounds herein are those having a solubility in water at 25° C. of less than about 1 g/100 g water, preferably less than about 0.5 g/100 g water, more preferably less than about 0.1 g/100 g water. Such water-insoluble oily compounds are selected from hydrocarbons, fatty compounds, and mixtures thereof. Such hydrocarbons include, for example, poly α-olefin oils, paraffins, waxes, and mixtures thereof. Such fatty compounds include, for example, fatty alcohols such as cetyl alcohol and stearyl alcohol, fatty acids such as stearic acid, fatty alcohol derivatives and fatty acid derivatives such as esters and ethers thereof, and mixtures thereof.

Preferably the composition of the present invention is transparent or translucent, and more preferably transparent. In the present invention, the composition being “transparent” means that the composition has a transmittance of about 50% or more, preferably about 65% or more, more preferably about 80% or more. In the present invention, the composition being “translucent” means that the composition has a transmittance of from about 25% to about 50%, preferably from about 35% to about 50%. The transmittances are measured at 600 nm using UV-1601 which is a UV-visible spectrophotometer available from Shimadzu.

Preferably, the composition is substantially free of anionic compounds. Anionic compounds herein include anionic surfactants and anionic polymers. In the present invention, the composition being “substantially free of anionic compounds” means that the composition includes 1% or less, preferably 0.5% or less, more preferably 0% of anionic compounds.

Hydrophobically Modified Cationic Thickening Polymer

The conditioning composition of the present invention comprises a hydrophobically modified cationic thickening polymer. The thickening polymers useful herein are those which can provide appropriate viscosity and rheology properties to the composition, so that the composition of the present composition has a suitable viscosity, preferably from about 100 cps to about 100,000 cps, more preferably from about 1,000 cps to about 50,000 cps, still more preferably from about 2,000 cps to about 50,000 cps, even more preferably from about 5,000 cps to about 20,000 cps. The viscosity herein can be suitably measured by Brookfield RVT at a shear rate of 2·s⁻¹ at 26.7° C.

Preferably, the hydrophobically modified cationic thickening polymers useful herein is hydrophilic, and has a solubility in water at 25° C. of at least 0.25 g/100 g water, more preferably at least 2 g/100 g water, still more preferably at least 5 g/100 g water, even more preferably at least 15 g/100 g water.

The thickening polymers are included in the composition of the present invention at a level by weight of from about 0.01% to about 5.0%, preferably from about 0.1% to about 3.0%, more preferably from about 0.3% to about 2.0%, still more preferably from about 0.4% to about 0.8%.

The hydrophobically modified cationic thickening polymer useful herein comprises a hydrophilic polymer backbone and a hydrophobic substitution group. The hydrophobically modified cationic thickening polymer useful herein also comprises a cationic substitution group.

The hydrophilic polymer backbone useful herein include, for example, a hydrophilic cellulose, a hydrophilic guar gum, a hydrophilic synthetic polymer, and mixtures thereof. The hydrophilic cellulose useful herein include, for example, hydroxymethyl cellulose, hydroxyethyl cellulose, hydroxyethyl ethylcellulose, hydroxypropyl cellulose, hydroxypropyl methylcellulose, hydroxybutyl cellulose, and mixtures thereof. The hydrophilic synthetic polymer useful herein include, for example, those containing hydrophilic monomers. Such monomers useful herein include, for example, those having polyalkylene oxide groups containing from about 1 to about 50 alkylene oxides, preferably from about 5 to about 45, more preferably from about 10 to about 40. The alkylene oxides are preferably selected from ethylene oxides, propylene oxides, and mixtures thereof. Preferred are those selected from the group consisting of vinyl alkoxylates, PEG 10-40 (meth)acrylates, and mixtures thereof. Among the above hydrophilic polymer backbones, preferred is hydroxyethyl cellulose.

The hydrophobically modified cationic thickening polymer further comprises a cationic substitution group. Preferred cationic substitution group useful herein has the following formula (i):

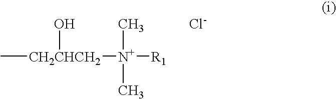

wherein R₁ is an alkyl having from about 1 to about 7 carbon atoms, preferably from about 1 to about 3 carbon atoms, more preferably R₁ is methyl.

The hydrophilic polymer backbone is further substituted with a hydrophobic substitution group. Preferred hydrophobic substitution group useful herein is cationic and has the following formula (ii):

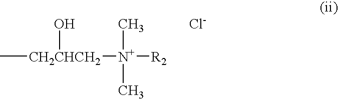

wherein R₂ is an alkyl group having from about 8 to about 22 carbon atoms, preferably from about 10 to about 18 carbon atoms, more preferably R₂ is an alkyl group having 12 carbon atoms.

The hydrophobically modified cationic thickening polymers useful herein have a molecular weight of preferably from about 50,000 to about 10,000,000, more preferably from about 75,000 to about 2,000,000, still more preferably from about 100,000 to about 500,000. The hydrophobically modified cationic thickening polymers useful herein have a cationic charge density of preferably from about 0.1 meq/g to about 2.0 meq/g, more preferably from about 0.3 meq/g to about 1.5 meq/g, still more preferably from about 0.5 to about 1.2 meq/g. The cationic charge can result from both of the cationic substitution group having the formula (i) and the hydrophobic substitution group having the formula (ii). The hydrophobically modified cationic thickening polymers useful herein also have a level of hydrophobic substitution of preferably from about 0.05 to about 0.15, more preferably from about 0.07 to about 0.15, still more preferably from about 0.10 to about 0.12. By “a level of hydrophobic substitution”, what is meant is mols of hydrophobic substitutions (corresponding to R₂ group in the formula (ii) above) per one glucose, one mannose, or a hydrophilic systhetic monomer unit. Especially, when the hydrophobically modified cationic thickening polymers have a molecular weight of less than about 500,000, they have a level of hydrophobic substitution of preferably from about 0.10 to about 0.15, more preferably from about 0.10 to about 0.12.

The highly preferred hydrophobically modified cationic thickening polymer of the present invention has both the cationic substitution group having the formula (i) and the hydrophobic substitution group having the formula (ii). By these substitution groups, the hydrophobically modified cationic thickening polymers of the present invention have a higher cationic charge density and a higher hydrophobic substitution level. It is believed that; among thickening polymers of the above formula, those having a higher molecular weight provide more viscous composition, those having a higher cationic charge density provide less viscous composition, and those having a higher hydrophobic substitution provide more viscous composition. It is also believed that; by the selection of molecular weight, cationic charge density, and hydrophobic substitution level, the thickening polymer of the present invention can provide balanced viscosity and rheology suitable for conditioning compositions.

The composition of the present invention can contain a mixture of hydrophobically modified cationic thickening polymers. Preferably, the mixture contains a first hydrophobically modified cationic thickening polymer having a molecular weight of from about 50,000 to about 500,000, and a second hydrophobically modified cationic thickening polymer having a molecular weight of from about 500,000 to about 10,000,000. Such mixture can provide a balanced benefit in view of rheology and conditioning properties, at a lower total level of thickening polymers.

A preferred hydrophobically modified cationic thickening polymer is a hydrophobically modified cationic hydroxyethyl cellulose polymer having the following formula:

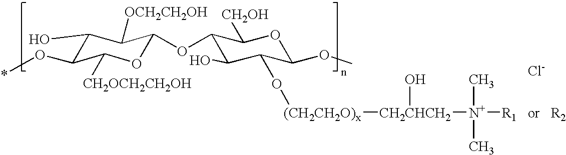

wherein R₁ and R₂ are defined above; n is an integer of from 200 to about 7,000, preferably from about 250 to about 5,000; x is 0 or an integer of from 1 to about 6, preferably from about 1 to about 3. Especially preferred hydrophobically modified cationic thickening polymers useful herein include, for example, a hydrophobically modified cationic cellulose available from Amerchol.

Surfactant

The compositions of the present invention comprise a surfactant. The surfactant is included in the composition at a level by weight of from about 0.05% to about 5%, preferably from about 0.1% to about 3%, more preferably from about 0.2% to about 2%, still more preferably from about 0.3 to about 1%.

Preferably, in view of the desire for a transparent or translucent appearance, the surfactant system is substantially soluble in the composition at the level used. By “substantially soluble” surfactant system, what is meant is that the composition has a transmittance of 50% or more, preferably about 65% or more, more preferably about 80% or more at 25° C. when containing the surfactant system at the level used.

The surfactant useful herein is selected from the group of consisting of a cationic surfactant, a non ionic surfactant, and mixtures thereof.

A variety of cationic surfactants which can be used in the composition of the present invention are described below. Among them, preferred are mono-long alkyl trimethyl ammonium salts. The mono-long alkyl trimethyl ammonium salts useful herein are those in which the alkyl has from 12 to 28 carbon atoms, preferably from 16 to 22 carbon atoms. Highly preferred mono-long alkyl trimethyl ammonium salts are, for example, cetyl trimethyl ammonium chloride, stearyl trimethyl ammonium chloride.

Cationic surfactants useful herein include, for example, those corresponding to the general formula (I):

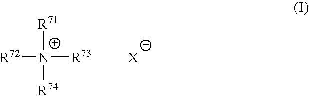

wherein at least one of R⁷¹, R⁷², R⁷³ and R⁷⁴ is selected from an aliphatic group of from 8 to 30 carbon atoms or an aromatic, alkoxy, polyoxyalkylene, alkylamido, hydroxyalkyl, aryl or alkylaryl group having up to about 22 carbon atoms, the remainder of R⁷¹, R⁷², R⁷³ and R⁷⁴ are independently selected from an aliphatic group of from 1 to about 22 carbon atoms or an aromatic, alkoxy, polyoxyalkylene, alkylamido, hydroxyalkyl, aryl or alkylaryl group having up to about 22 carbon atoms; and X is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferred is wherein R⁷¹, R⁷², R⁷³ and R⁷⁴ are independently selected from C₁ to about C₂₂ alkyl. Nonlimiting examples of cationic surfactants useful in the present invention include the materials having the following CTFA designations: quaternium-8, quaternium-14, quaternium-18, quaternium-18 methosulfate, quaternium-24, and mixtures thereof.

Among the cationic surfactants of general formula (I), preferred are those containing in the molecule at least one alkyl chain having at least 16 carbons. Nonlimiting examples of such preferred cationic surfactants include: behenyl trimethyl ammonium chloride available, for example, with tradename Genamine KDMP from Clariant, with tradename INCROQUAT TMC-80 from Croda, and with tradename ECONOL TM22 from Sanyo Kasei; cetyl trimethyl ammonium chloride available, for example, with tradename CTAC 30KC from KCI, and with tradename CA-2350 from Nikko Chemicals; stearyl trimethyl ammonium chloride available, for example, with tradename Genamine STACP from Clariant; olealkonium chloride available, for example, with tradename Incroquat O-50 from Croda; hydrogenated tallow alkyl trimethyl ammonium chloride, dialkyl(14-18)dimethyl ammonium chloride, ditallow alkyl dimethyl ammonium chloride, dihydrogenated tallow alkyl dimethyl ammonium chloride, distearyl dimethyl ammonium chloride, dicetyl dimethyl ammonium chloride, di(behenyl/arachidyl) dimethyl ammonium chloride, dibehenyl dimethyl ammonium chloride, stearyl dimethyl benzyl ammonium chloride, stearyl propyleneglycol phosphate dimethyl ammonium chloride, stearoyl amidopropyl dimethyl benzyl ammonium chloride, stearoyl amidopropyl dimethyl (myristylacetate)ammonium chloride, and N-(stearoyl colamino formyl methyl) pyridinium chloride.

Also preferred are hydrophilically substituted cationic surfactants in which at least one of the substituents contain one or more aromatic, ether, ester, amido, or amino moieties present as substituents or as linkages in the radical chain, wherein at least one of the R71-R74 radicals contain one or more hydrophilic moieties selected from alkoxy (preferably C1-C3 alkoxy), polyoxyalkylene (preferably C1-C3 polyoxyalkylene), alkylamido, hydroxyalkyl, alkylester, and combinations thereof. Preferably, the hydrophilically substituted cationic conditioning surfactant contains from 2 to about 10 nonionic hydrophile moieties located within the above stated ranges. Highly preferred hydrophilically substituted cationic surfactants include dialkylamido ethyl hydroxyethylmonium salt, dialkylamidoethyl dimonium salt, dialkyloyl ethyl hydroxyethylmonium salt, dialkyloyl ethyldimonium salt, and mixtures thereof; for example, commercially available under the following tradenames; VARISOFT 110, VARISOFT 222, VARIQUAT K1215 and VARIQUAT 638 from Witco Chemical, MACKPRO KLP, MACKPRO WLW, MACKPRO MLP, MACKPRO NSP, MACKPRO NLW, MACKPRO WWP, MACKPRO NLP, MACKPRO SLP from McIntyre, ETHOQUAD 18/25, ETHOQUAD O/12PG, ETHOQUAD C/25, ETHOQUAD S/25, and ETHODUOQUAD from Akzo, DEHYQUAT SP from Henkel, and ATLAS G265 from ICI Americas. Babassuamidopropalkonium Chloride available from Croda under the tradename Incroquat BA-85 is also preferably used in the composition.

Amines are suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoetlhyldiethylamine, arachidamidoethyldimethylamine, diethylaminoethylstearamide. Also useful are dimethylstearamine, dimethylsoyamine, soyamine, myristylamine, tridecylamine, ethylstearylamine, N-tallowpropane diamine, ethoxylated (with 5 moles of ethylene oxide) stearylamine, dihydroxyethylstearylamine, and arachidylbehenylamine. Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

A variety of nonionic surfactants can be used in the composition of the present invention. Such nonionic surfactants include, for example, polyoxyethylene alkyl ethers; polysorbates such as polysorbate-20 through 80; polyethylene glycol derivatives of glycerides such as polyethylene glycol derivatives of hydrogenated castor oil including PEG-40 through 100 hydrogenated castor oil, and polyethylene glycol derivatives of stearic acid including PEG-10 through 55 stearate. Among a variety of nonionic Surfactants, preferred are polyoxyethylene alkyl ethers. Especially preferred polyoxyethylene alkyl ethers useful herein are those having a C12-C22 alkyl chain, and having the above HLB value. Highly preferred polyoxyethylene alkyl ethers include, for example, laureth series of compounds such as laureth-7 through 12; ceteth series of compounds such as ceteth-7 through 20; and pareth series of compounds such as pareth-9 through 15.

Hydrophobically Modified Silicone

The compositions of the present invention comprise a hydrophobically modified silicone. The hydrophobically modified silicone copolyol is included in the composition at levels by weight of from about 0.1% to about 10%, more preferably from about 0.5% to about 8%, still more preferably from about 1% to about 6%, even more preferably from about 2% to about 5%.

Among a variety of silicone compounds, preferred are those selected from the group consisting of (i) silicone nanoemulsion having an average particle size of 300 nm or less, preferably 200 nm or less, more preferably 100 nm or less when contained in the composition, (ii) silicone compound being substantially soluble in the composition, and (iii) mixtures thereof, in view of the desire for a transparent or translucent appearance. By “substantially soluble” silicone compound, what is meant is that the composition has a transmittance of 50% or more, preferably about 65% or more, more preferably about 80% or more at 25° C. when containing the silicone compound at the level used.

Commercially available silicone nanoemulsion useful herein includes, for example, that with a tradename Silicone DC-8177 available from Dow Corning; quaternized silicone nanoemulsion with a tradename DC5-7133 available from Dow Corning; and amodimethicone nanoemulsion with a tradename XS65-B6413 available from General Electric.

With respect to substantially soluble silicone compounds, for example, following materials can be substantially soluble depending on the level of hydrophilic groups in their structure: silicone copolyols such as dimethicone copolyol with a tradename Silicone DC-5330 from Dow Corning; amino silicone copolyols such as those having an INCI name Bis(C13-15 Alkoxy) PG Amodimethicone available with a tradename DC2-8500 from Dow Corning; hydrophobically modified amino silicone copolyols; hydrophobically modified amido silicone copolyols; and quaternized silicones. Among these substantially soluble silicone compounds, more preferred are those selected from the group consisting of amino silicone copolyols, hydrophobically modified amino silicone copolyols, and hydrophobically modified amido silicone copolyols, and mixtures thereof, still more preferred are hydrophobically modified amido silicone copolyols in view of providing improved silicone deposition thus providing improved conditioning performance while meeting the desire for transparent or translucent appearance. Such hydrophobically modified amidomethicone copolyols have the following formula:

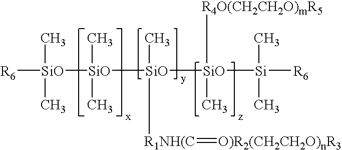

wherein R₁, R₂, R₄ are respectively C1-C3 alkyl, preferably ethyl; R₃ is an alkyl group having 8-22 carbon atoms, preferably 10-20 carbon atoms, more preferably 12-16 carbon atoms, even more preferably 12 carbon atoms; R₅ is H or C1-C3 alkyl, preferably methyl; R₆ is OH or CH₃, preferably methyl; n is an integer of 1-10, highly preferably 5; m is an integer of 2-20, highly preferably 12; n+m=3-30, preferably 5-25, more preferably 8-20, even more preferably 17; x is an integer from 200 to 500, preferably from 300 to 400; y is an integer from 5 to 40, preferably from 10 to 30; and z is 0 or an integer from 1 to 30, preferably from 5 to 20. Commercially available hydrophobically modified amido silicone copolyols are, for example, those having an INCI name PEG-12 Methyl Ether/Lauroxy PEG-5 Amidopropyl Dimethicone available with a tradename Silicone BY16-906 from Dow Corning.

In view of providing improved conditioning benefits, it is preferred for the composition of the present invention to provide improved silicone deposition, even after rinsing-off the composition from the hair. For example, it is preferred for the compositions to provide silicone deposition of about 50 ppm or more, more preferably about 100 ppm or more, still more preferably 200 ppm or more, even more preferably 400 ppm or more after rinsing-off the hair. The amount of the silicone deposition can be measured by a method consisting of: (i) a preparation of hair switch; and (ii) silicone deposition measurement.

(i) Preparation of Hair Switch

For the silicone deposition measurement, 2 gram hair switches are used. The hair switches are prepared by following steps:

(a) Providing five cycles of shampoo/conditioning treatments to the hair switch, each cycle of shampoo/conditioning treatment consisting of following steps:

(a-1) Applying a shampoo at a level of 0.2 cc and lathering the hair switch; and rinsing the hair switch;

(a-2) Applying a shampoo again at a level of 0.2 cc and lathering the hair switch; and rinsing the hair switch; and

(a-3) Then providing conditioner treatment to the hair switch, the conditioner treatment consisting of applying a conditioner at a level of 0.2 cc and treating the hair switch; and rinsing the hair switch; and

(b) Then drying the hair switch.

The hair switch is ready for the measurement of its silicone deposition amount.

(ii) Silicone Deposition Measurement

The deposited silicone on the hair switch is extracted in an appropriate solvent. The extracts are then introduced into an atomic absorption/emission detector instrument and measured at the appropriate wavelength. The absorbance/emission value returned by the instrument is then converted to actual concentration (ppm) of silicone compound deposited on the hair through an external calibration curve obtained with known weights of a well characterized standard of the silicone compound under study.

Aqueous Carrier

The compositions of the present invention comprise an aqueous carrier. The level and species of the carrier are selected according to the compatibility with other components, and other desired characteristic of the product.

Carriers useful in the present invention include water and water solutions of lower alkyl alcohols. Lower alkyl alcohols useful herein are monohydric alcohols having 1 to 6 carbons, more preferably ethanol and isopropanol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 40% to about 98%, and more preferably from about 50% to about 98% water.

The pH of the present composition is preferably from about 3 to about 9, more preferably from about 3 to about 7, still more preferably from 4 to 6. Buffers and other pH adjusting agents call be included to achieve the desirable pH.

Additional Components

The composition of the present invention may include additional components, which may be selected by the artisan according to the desired characteristics of the final product and which are suitable for rendering the composition more cosmetically or aesthetically acceptable or to provide them with additional usage benefits.

Humectant and/or Co-Solvent

The compositions of the present invention may contain a humectant and/or co-solvent to help the surfactant system and/or silicone compound to be substantially soluble in the composition. The humectants and/or co-solvents herein are selected from the group consisting of polyhydric alcohols, water soluble alkoxylated nonionic polymers, and mixtures thereof. The humectants and/or co-solvents herein are preferably used at levels by weight of the composition of from about 0.1% to about 20%, more preferably from about 0.5% to about 5%.

Polyhydric alcohols useful herein include glycerin, sorbitol, propylene glycol, butylene glycol, hexylene glycol, ethoxylated glucose, 1,2-hexane diol, hexanetriol, dipropylene glycol, erythritol, trehalose, diglycerin, xylitol, maltitol, maltose, glucose, fructose, sodium chondroitin sultate, sodium hyaluronate, sodium adenosin phosphate, sodium lactate, pyrrolidone carbonate, glucosamine, cyclodextrin, and mixtures thereof. Among them, preferred for the co-solvents are 1,2-hexane diol, hexylene glycol, butylene glycol, glycerine, and mixtures thereof.

Water soluble alkoxylated nonionic polymers useful herein include polyethylene glycols and polypropylene glycols having a molecular weight of up to about 10,000 such as those with CTFA names PEG-4, PEG-8, PEG-12, PEG-20, PEG-150 and mixtures thereof.

Additional Silicone Components

The composition of the present invention may contain additional silicone components. Such additional silicone components include, for example, polyalkyl siloxanes such as polydimethylsiloxane from General Electric Company in their TSF 451 series and from Dow Corning in their Dow Corning SH200 series; polyaryl siloxanes; polyalkylaryl siloxanes; polyether siloxane copolymers; amino substituted silicones such as amodimethicone with tradename BY16-872 available from Dow Corning; silicone microemulsion with tradename DC2-8194 available from Dow Corning; quaternized silicones such as that available from Union Carbide under the tradename UCAR SILICONE ALE 56 and that available from Noveon with a tradename Ultrasil Q-Plus; and mixtures thereof.

Additional Thickening Polymer

The compositions of the present invention can contain additional thickening polymers. The additional thickening polymers useful herein are those which can provide appropriate viscosity and rheology properties to the composition, so that the composition of the present composition has a suitable viscosity.

The additional thickening polymer can be included in the composition of the present invention at a level by weight of from about 0.01% to about 5%, still more preferably from about 0.05% to about 3%, even more preferably from about 0.1% to about 2.0%.

A variety of additional thickening polymers other than those described above under the title “HYDROPHOBICALLY MODIFIED CATIONIC THICKENING POLYMER” can be used in the compositions of the present invention. Additional thickening polymers useful herein include, for example, cellulose and its derivatives such as hydroxymethyl cellulose, hydroxyethyl cellulose, hydroxyethylethylcellulose, cetyl hydroxyethylcellulose having tradenames NATROSOL PLUS 330CS and POLYSURF 67, both available from Aqualon Company, Del, USA; guar gums such as cationic or nonionic guar gums; crosslinked polymers such as nonionic crosslinked polymers and cationic crosslinked polymers; and acrylate polymers such as sodium polyacryl ate, polyethylacrylate, and polyacrylamide; and Polyquaternium-37 available from 3V Sigma with tradenames Synthalen CR, Synthalen CU, and Synthalen CN. The thickening polymers useful herein may include the polymers disclosed below under the title “Cationic conditioning polymer”.

Cationic Conditioning Polymer

The conditioning compositions of the present invention may further include cationic conditioning polymers. The cationic polymers hereof will generally have a weight average molecular weight which is at least about 5,000, typically at least about 10,000, and is less than about 10 million, preferably, the molecular weight is from about 100,000 to about 2 million. The cationic polymers useful herein may include the polymers disclosed above under the title “Additional thickening polymer”.

The cationic conditioning polymer can be included in the compositions at a level by weight of preferably from about 0.01% to about 10%, more preferably from about 0.05% to about 5%.

Suitable cationic conditioning polymers include, for example: copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquaternium-16), such as those commercially available from BASF Wyandotte Corp. (Parsippany, N.J., USA) under the LUVIQUAT tradename (e.g., LUVIQUAT FC 370); copolymers of 1-vinyl-2-pyrrolidone and dimethylaminoethyl methacrylate (referred to in the industry by CTFA as Polyquaternium-11) such as those commercially available from Gaf Corporation (Wayne, N.J., USA) under the GAFQUAT tradename (e.g., GAFQUAT 755N); cationic diallyl quaternary ammonium-containing polymers, including, for example, dimethyldiallylammonium chloride homopolymer and copolymers of acrylamide and dimethyldiallylammonium chloride, referred to in the industry (CTFA) as Polyquaternium 6 and Polyquaternium 7, Polyquaternium-7 including that commercially available with a tradename Merquat 550 from Ondeo Nalco; polymethacrylamidopropyl trimonium chloride such as that commercially available with a tradename Polycare 133 from Rhone-Poulenc; and Polyquaternium-37 available from 3V Sigma with tradenames Synthalen CR, Synthalen CU, and Synthalen CN.

Also suitable cationic conditioning polymers herein include cationic cellulose derivatives. Cationic cellulose derivative useful herein include, for example, salts of hydroxyethyl cellulose reacted with trimethyl ammonium substituted epoxide, referred to in the industry (CTFA) as Polyquaternium 10, available from Amerchol Corp. (Edison, N.J., USA) in their Polymer JR® and LR® series, and also available from National Starch & Chemical with a tradename Celquat SC-230M; and Polyquaternium-4 with tradename Celquat H-100 available from National Starch & Chemical.

Other suitable cationic conditioning polymers include cationic guar gum derivatives, such as guar hydroxypropyltrimonium chloride commercially available from Rhodia in their Jaguar series.

Other Additional Components

The compositions of the present invention may further include other additional components. Other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, nonionic surfactants such as glyceryl stearate available from Stepan Chemicals, hydrolysed keratin, proteins, plant extracts, and nutrients; emollients such as PPG-3 myristyl ether with tradename Varonic APM available from Goldschmidt, Trimethyl pentanol hydroxyethyl ether, PPG-11 stearyl ether with tradename Varonic APS available from Goldschmidt, Stearyl heptanoate with tradename Tegosoft SH available from Goldschmidt, Lactil (mixture of Sodium lactate, Sodium PCA, Glycine, Fructose. Urea, Niacinamide, Inositol, Sodium Benzoate, and Lactic acid) available from Goldschmidt, Ethyl hexyl palmitate with tradename Saracos available from Nishin Seiyu and with tradename Tegosoft OP available from Goldschmidt; hair-fixative polymers such as amphoteric fixative polymers, cationic fixative polymers, anionic fixative polymers, nonionic fixative polymers, and silicone Drafted copolymers; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; hair oxidizing (bleaching) agents, such as hydrogen peroxide, perborate and persulfate salts; hair reducing agents such as the thioglycolates; perfumes; and sequestering agents, such as disodium ethylenediamine tetra-acetate; ultraviolet and infrared screening and absorbing agents such as octyl salicylate; antidandruff agents such as zinc pyrrithione and salicylic acid; visible particles with tradenames Unisphere and Unicerin available from Induchem AG (Switzerland); and anti-foaming agent such as that with a tradename XS63-B8929 available from GE-Toshiba Silicone.

Product Forms

The conditioning compositions of the present invention can be in the form of rinse-off products or leave-on products, can be transparent, translucent, or opaque, and can be formulated in a wide variety of product forms, including but not limited to creams, gels, emulsions, mousses and sprays.

The conditioning compositions of the present invention can be used for conditioning hair and/or skin by applying the compositions to the hair and/or skin. The conditioning composition of the present invention is especially suitable for hair care products such as hair conditioners and skin care products such as skin conditioners.

The conditioning compositions of the present invention are especially suitable for hair conditioners for rinse-off use. Such compositions are preferably used by following steps:

(i) after shampooing hair, applying to the hair an effective amount of the conditioning composition for conditioning the hair; and

(ii) then rinsing the hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Ingredients are identified by chemical or CTFA name, or otherwise defined below.

Compositions (wt %)

| | Ex.1 | Ex.2 | Ex.3 | Ex.4 | Ex.5 | Ex.6 | Ex.7 |
|---|---|---|---|---|---|---|---|
| Hydrophobically modified cationic | 0.53 | 0.8 | 0.7 | 0.6 | 0.63 | — | 0.4 |
| cellulose polymer-1 *1 | | | | | | | |
| Hydrophobically modified cationic | — | — | — | — | — | 1.5 | — |
| cellulose polymer-2 *2 | | | | | | | |
| Hydrophobically modified cationic | — | — | — | — | — | — | 0.3 |
| cellulose polymer-6 *6 | | | | | | | |
| Cetyl trimethyl ammonium chloride *10 | 0.65 | 0.5 | — | 0.8 | 0.6 | 0.7 | 0.8 |
| Stearyl trimethyl ammonium chloride | — | — | 0.6 | — | — | — | — |
| *11 | | | | | | | |
| Hydrophobically substituted | 4.0 | 5.0 | 4.5 | — | 4.0 | 3.5 | 5.0 |
| amidomethicone copolyol *13 | | | | | | | |
| Hydrophobically substituted | — | — | — | 5.0 | 2.0 | — | — |
| aminomethicone copolyol *14 | | | | | | | |
| Perfume | 0.3 | 0.3 | 0.3 | 0.3 | 0.3 | 0.3 | 0.3 |
| Disodium EDTA | 0.13 | 0.13 | 0.13 | 0.13 | 0.13 | 0.13 | 0.13 |
| Methyl Paraben | 0.2 | 0.2 | 0.2 | 0.2 | 0.2 | 0.2 | 0.2 |
| Methylchloroisothiazolinone/ | 0.033 | 0.033 | 0.033 | 0.033 | 0.033 | 0.033 | 0.033 |
| Methylisothiazolinone *15 | | | | | | | |
| Deionized Water | - - - q.s. to 100% - - - | | | | | | |
| | Ex.8 | Ex.9 | Ex.10 | Ex.11 | Ex.12 | Ex.13 | Ex.14 |
| Hydrophobically modified cationic | 0.5 | — | — | — | — | — | — |
| cellulose polymer-1 *1 | | | | | | | |
| Hydrophobically modified cationic | 0.2 | 2 | 0.7 | 0.63 | — | — | 0.3 |
| cellulose polymer-3 *3 | | | | | | | |
| Hydrophobically modified cationic | — | — | — | — | 1.2 | 0.4 | — |
| cellulose polymer-4 *4 | | | | | | | |
| Hydrophobically modified cationic | — | — | — | — | — | — | 0.2 |
| cellulose polymer-6 *6 | | | | | | | |
| Cetyltrimethylammoniumchloride *10 | 0.6 | 1.5 | — | — | 0.8 | 0.6 | 0.75 |
| Stearyl trimethyl ammoniumchloride | — | — | 0.55 | — | — | — | — |
| *11 | | | | | | | |
| Laureth-9 *12 | — | — | — | 0.7 | — | — | — |
| Hydrophobically substituted | 3 | 4 | 4.5 | 5 | — | 5 | — |
| amidomethicone copolyol *13 | | | | | | | |
| Hydrophobically substituted | — | — | — | — | 6 | — | 4 |
| aminomethicone copolyol *14 | | | | | | | |
| Perfume | 0.3 | 0.3 | 0.3 | 0.3 | 0.3 | 0.3 | 0.3 |
| Disodium EDTA | 0.13 | 0.13 | 0.13 | 0.13 | 0.13 | 0.13 | 0.13 |
| Methyl Paraben | 0.2 | 0.2 | 0.2 | 0.2 | 0.2 | 0.2 | 0.2 |
| Methylchloroisothiazolinone/ | 0.033 | 0.033 | 0.033 | 0.033 | 0.033 | 0.033 | 0.033 |
| Methylisothiazolinone *15 | | | | | | | |
| Deionized Water | - - - q.s. to 100% - - - | | | | | | |
| | Ex.15 | Ex.16 | Ex.17 | Ex.18 | Ex.19 | Ex.20 | Ex.21 |
| Hydrophobically modified cationic | 0.7 | — | — | — | — | — | — |
| cellulose polymer-5 *5 | | | | | | | |
| Hydrophobically modified cationic | — | 0.4 | 0.3 | — | — | — | — |
| cellulose polymer-6 *6 | | | | | | | |
| Hydrophobically modified cationic | — | — | — | 0.65 | — | — | — |
| cellulose polymer-7 *7 | | | | | | | |
| Hydrophobically modified cationic | — | — | — | — | 0.9 | 0.5 | — |
| guar polymer-1 *8 | | | | | | | |
| Hydrophobically modified cationic | — | — | — | — | — | 0.5 | 1 |
| guar polymer-2 *9 | | | | | | | |
| Cetyltrimethylammoniumchloride *10 | — | — | 0.65 | 0.7 | 0.7 | 0.6 | 0.9 |
| Stearyl trimethyl ammonium chloride | 0.6 | 0.7 | — | — | — | — | — |
| *11 | | | | | | | |
| Hydrophobically substituted | 5 | 5 | 4 | 4 | 5 | 4 | 6 |
| amidomethicone copolyol *13 | | | | | | | |
| Perfume | 0.3 | 0.3 | 0.3 | 0.3 | 0.3 | 0.3 | 0.3 |
| Disodium EDTA | 0.13 | 0.13 | 0.13 | 0.13 | 0.13 | 0.13 | 0.13 |
| Methyl Paraben | 0.2 | 0.2 | 0.2 | 0.2 | 0.2 | 0.2 | 0.2 |
| Methylchloroisothiazolinone/ | 0.033 | 0.033 | 0.033 | 0.033 | 0.033 | 0.033 | 0.033 |
| Methylisothiazolinone *15 | | | | | | | |
| Deionized Water | - - - q.s. to 100% - - - | | | | | | |
| | Ex.22 | Ex.23 | Ex.24 | Ex.25 | Ex.26 | Ex.27 | Ex.28 |
| Hydrophobically modified cationic | 0.8 | 0.6 | — | — | 0.3 | 0.6 | 0.8 |
| cellulose polymer-1 *1 | | | | | | | |
| Hydrophobically modified cationic | — | — | 1.5 | — | — | — | — |
| cellulose polymer-2 *2 | | | | | | | |
| Hydrophobically modified cationic | — | — | — | 2.5 | — | — | — |
| cellulose polymer-3 *3 | | | | | | | |
| Hydrophobically modified cationic | — | — | — | — | 0.3 | — | — |
| cellulose polymer-6 *6 | | | | | | | |
| Cetyltrimethylammonium chloride *10 | 0.5 | 0.4 | 0.7 | 1.5 | 0.8 | 0.7 | — |
| Stearyl trimethyl ammonium chloride | — | 0.45 | — | — | — | — | 0.4 |
| *11 | | | | | | | |
| Hydrophobically substituted | 4.5 | — | 4.0 | 4.0 | 4.0 | 4.0 | 3.0 |
| amidomethicone copolyol *13 | | | | | | | |
| Hydrophobically substituted | — | 5.0 | 2.0 | — | — | — | — |
| aminomethicone copolyol *14 | | | | | | | |
| Quaternized silicone nanoemulsion | — | — | — | — | — | 1.5 | — |
| *16 | | | | | | | |
| Silicone microemulsion *17 | — | — | — | — | — | — | 2.0 |
| 1,2-hexanediol | 1.0 | — | — | — | 0.5 | — | 1.0 |
| Butylene glycol | — | — | — | — | 0.5 | — | — |
| PEG-12 *18 | — | — | — | 1.0 | — | — | — |
| PEG-200 *19 | — | — | 1.0 | — | — | — | — |
| Perfume | 0.3 | 0.3 | 0.3 | 0.3 | 0.3 | 0.3 | 0.3 |
| Disodium EDTA | 0.13 | 0.13 | 0.13 | 0.13 | 0.13 | 0.13 | 0.13 |
| Methyl Paraben | 0.2 | 0.2 | 0.2 | 0.2 | 0.2 | 0.2 | 0.2 |
| Methylchloroisothiazolinone/ | 0.033 | 0.033 | 0.033 | 0.033 | 0.033 | 0.033 | 0.033 |
| Methylisothiazolinone *15 | | | | | | | |

| | |
|---|---|
| Deionized Water | - - - q.s. to 100% - - - |

Definitions of Components
*1 Hydrophobically modified cationic cellulose polymer-1:
comprised by a hydroxyethyl cellulose backbone, a cationic substitution group having the formula (i), and a hydrophobic substitution group having the formula (ii):

(i)

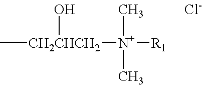

(ii)

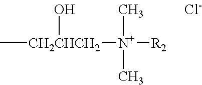

wherein R₁ is methyl and R₂ is an alkyl group having 12 carbon atoms; and having a molecular weight of about 420,000, a cationic charge density about 0.8 meq/g and a level of hydrophobic substitution about 0.115
*2 Hydrophobically modified cationic cellulose polymer-2:
comprised by the same backbone and substitution groups as those for the polymer-1, and having a molecular weight of about 470,000, a cationic charge density about 1.8 meq/g and a level of hydrophobic substitution about 0.115
*3 Hydrophobically modified cationic cellulose polymer-3:
comprised by the same backbone and substitution groups as those for the polymer-1, and having a molecular weight of about 1,300,000, a cationic charge density about 0.8 meq/g and a level of hydrophobic substitution about 0.115
*4 Hydrophobically modified cationic cellulose polymer-4:
comprised by the same backbone and substitution groups as those for the polymer-1, and having a molecular weight of about 1,500,000, a cationic charge density about 1.8 meq/g and a level of hydrophobic substitution about 0.115
*5 Hydrophobically modified cationic cellulose polymer-5:
comprised by the same backbone and substitution groups as those for the polymer-1, and having a molecular weight of about 2,200,000, a cationic charge density about 0.8 meq/g and a level of hydrophobic substitution about 0.10
*6 Hydrophobically modified cationic cellulose polymer-6:
comprised by the same backbone and substitution groups as those for the polymer-1, and having a molecular weight of about 2,500,000, a cationic charge density about 1.8 meq/g and a level of hydrophobic substitution about 0.115
*7 Hydrophobically modified cationic cellulose polymer-7:
comprised by the same backbone and substitution groups as those for the polymer-1, and having a molecular weight of about 2,100,000, a cationic charge density about 0.3 meq/g and a level of hydrophobic substitution about 0.075
*8 Hydrophobically modified cationic guar polymer-1:
comprised by a guar backbone and the same substitution groups as those for the cellulose polymer-1, and having a molecular weight of about 2,200,000, a cationic charge density about 1.2 meq/g and a level of hydrophobic substitution about 0.05
*9 Hydrophobically modified cationic guar polymer-2:
comprised by the same backbone and substitution groups as those for the guar polymer-1, and having a molecular weight of about 400,000, a cationic charge density about 0.8 meq/g and a level of hydrophobic substitution about 0.10
*10 Cetyl trimethyl ammonium chloride:
CTAC 30KC available from KCI
*11 Stearyl trimethyl ammonium chloride:
Genamine STACP available from Clariant
*12 Laureth-9:
BL-9EX available from Nikko Chemicals
*13 Hydrophobically modified amidomethicone copolyol:
Silicone BY16-906 available from Dow Corning having the following formula:

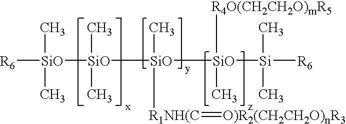

wherein R₁, R₂, and R₄ are ethyl; R₃ is an alkyl group having 12 carbon atoms; R₅ and R₆ are methyl; n is an integer of 5; m is an integer of 12; n + m = 17; x is an integer from 200 to 500; y is an integer from 5 to 40; and z is 0 or an integer from 1 to 30.
*14 Hydrophobically modified aminomethicone copolyol:
that having a following formula:

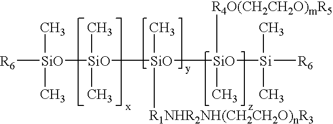

wherein R₁, R₂, R₃, R₄, R₅, R₆, n, m, n + m, x, y, and z are same as those defined above for “hydrophobically modified amidomethicone copolyol”.
*15 Methylchloroisothiazolinone/Methylisothiazolinone:
Kathon CG available from Rohm&Haas
*16 Quaternized silicone nanoemulsion:
DC5-7133 available from Dow Coming
*17 Silicone microemulsion:
DC2-8194 available from Dow Coming
*18 PEG-12:
Available from Dow Chemical
*19 PEG-200:
Emkapol available from ICI

Method of Preparation

The conditioning compositions of “Ex.1” to “Ex.28” as shown above can be prepared by any conventional method well known in the art. They are suitably made as follows:

The polymeric materials are dispersed in water at room temperature, mixed with vigorous agitation, and heated to 50-75° C. Silicones, and if included, cationic surfactants and other temperature insensitive components are added to the mixture with agitation. Then the mixture is cooled down to below 40° C., and then, if included, nonionic surfactants and the remaining components such as perfumes, preservatives, and anti-foaming agents, are added to the mixture with agitation.

Examples 1 through 28 are conditioning compositions of the present invention which are particularly useful for hair conditioners for rinse-off use. These examples have many advantages.

For example, the compositions of “Ex.1” through “Ex.28” have a suitable rheology for conditioning compositions, and provide conditioning benefits, especially softness and reduced tangling on wet hair when used for hair care products such as hair conditioning products. The compositions of “Ex.1” through “Ex.28” have transparent or translucent appearance. When used for hair care products, the composition of “Ex.1” through “Ex.28” can provide the above rheological and conditioning benefits while not weighing down the hair. When used in a form of rinse-off products, the compositions of “Ex.1” through “Ex.28” can provide easy to rinse-off feel while providing the above theological and conditioning benefits.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair or skin conditioning composition comprising by weight:
(a) from about 0.01% to about 5.0% of a hydrophobically modified cationic thickening polymer;
(b) from about 0.05% to about 10.0% of a surfactant selected from the group consisting of cationic surfactant, nonionic surfactant, and mixtures thereof;
(c) from about 0.1% to about 10% of a hydrophobically modified silicone; and
(d) an aqueous carrier.

2. The conditioning composition of claim 1 wherein the composition is substantially free of a water-insoluble high melting point oily compound.

3. The conditioning composition of claim 1 wherein the composition is transparent or translucent.

4. The conditioning composition of claim 1 wherein the composition is transparent.

5. The conditioning composition of claim 4 wherein the composition has a transmittance of 50% or more.

6. The conditioning composition of claim 5 wherein the composition has a transmittance of 65% or more.

7. The conditioning composition of claim 6 wherein the composition has a transmittance of 80% or more.

8. The conditioning composition of claim 1 wherein the hydrophobically modified cationic thickening polymer comprises a hydrophilic polymer backbone and a hydrophobic substitution group.

9. The conditioning composition of claim 8 wherein the hydrophilic polymer backbone is selected from the group consisting of a hydrophilic cellulose, a hydrophilic guar gum, a hydrophilic synthetic polymer, and mixtures thereof.

10. The conditioning composition of claim 8 wherein the hydrophobically modified cationic thickening polymer further comprises a cationic substitution group having the following formula (i): 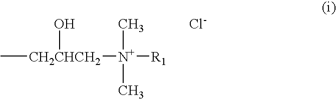
wherein R₁ is an alkyl having from about 1 to about 7 carbons.

11. The conditioning composition of claim 8 wherein the hydrophobic substitution group is cationic and has the following formula (ii): 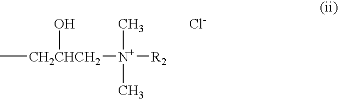
wherein R₂ is an alkyl group having from about 8 to about 22 carbons.

12. The conditioning composition of claim 8, wherein the hydrophobically modified cationic thickening polymer further comprises a cationic substitution group having the formula (i): 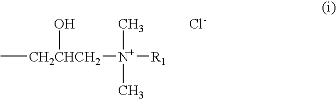
and further comprises a hydrophobic substitution group having the formula (ii):

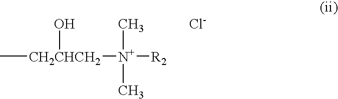

13. The conditioning composition of claim 12, wherein R₁ is an alkyl having 1 to about 3 carbon atoms, and R₂ is an alkyl group having from about 10 to about 18 carbon atoms.

14. The conditioning composition of claim 13 wherein R₁ is methyl and R₂ is an alkyl group having 12 carbon atoms.

15. The conditioning composition of claim 1 wherein the hydrophobically modified cationic thickening polymer has a molecular weight of less than about 500,000, and a level of hydrophobic substitution of from about 0.10 to about 0.15.

16. The conditioning composition of claim 1 wherein the hydrophobically modified silicone is selected from those having an average particle size of 300 nm or less in the composition, those being substantially soluble in the composition, and mixtures thereof.

17. The conditioning composition of claim 16 wherein the substantially soluble silicone compound is selected from the group consisting of amino silicone copolyols, hydrophobically modified amino silicone copolyols, hydrophobically modified amido silicone copolyols, and mixtures thereof.

18. The conditioning composition of claim 17 wherein the hydrophobically modified silicone is a hydrophobically modified amido silicone copolyol.

19. The conditioning composition of claim 18 wherein the hydrophobically modified amido silicone copolyol has the following formula: 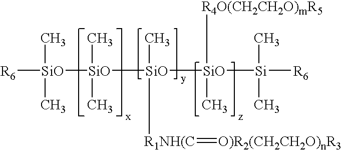
wherein R₁, R₂, R₄ are respectively C1-C3 alkyl; R₃ is an alkyl group having from about 8 to about 22 carbon atoms; R₅ is H or C1-C3 alkyl; R₆ is OH or CH₃; n is an integer of 1-10; m is an integer of 2-20; n+m=3-30; x is an integer from 200 to 500; y is an integer from 5 to 40; and z is 0 or an integer from 1 to 30.

20. The conditioning composition of claim 19 wherein; R₃ is an alkyl group having from about 10 to about 20 carbon atoms; n+m=5-25; x is an integer from 300 to 400; y is an integer from 10 to 30; and z is an integer from 5 to 20.

21. The conditioning composition of claim 20 wherein; R₃ is an alkyl group having from about 12 to about 16 carbon atoms; and n+m=8-20.

22. The conditioning composition of claim 19 wherein R₁, R₂, and R₄ are ethyl; R₃ is an alkyl group having 12 carbon atoms; R₅ and R₆ are methyl; n is an integer of 5; m is an integer of 12; and n+m=17.

23. The conditioning composition of claim 1 further comprising a co-solvent.

24. The conditioning composition of claim 23 wherein the co-solvent is selected from the group consisting of 1,2-hexane diol, hexylene glycol, butylene glycol, glycerine, and mixtures thereof.

25. The conditioning composition of claim 1 comprising by weight:
(a) from about 0.1% to about 3.0% of the hydrophobically modified cationic thickening polymer;
(b) from about 0.1% to about 3.0% of the surfactant;
(c) from about 0.5% to about 8.0% of the hydrophobically modified silicone; and
(d) an aqueous carrier.

26. The conditioning composition of claim 1 comprising by weight:
(a) from about 0.3% to about 2.0% of the hydrophobically modified cationic thickening polymer;
(b) from about 0.2% to about 2.0% of the surfactant;
(c) from about 0.1% to about 6.0% of the hydrophobically modified silicone; and
(d) an aqueous carrier.

27. The conditioning composition of claim 1 comprising by weight:
(a) from about 0.4% to about 0.8% of the hydrophobically modified cationic thickening polymer;
(b) from about 0.3% to about 2.0% of the surfactant;
(c) from about 2.0% to about 5.0% of the hydrophobically modified silicone; and
(d) an aqueous carrier.

28. The conditioning composition of claim 1 which is a hair conditioning composition.

29. The conditioning composition of claim 1 which is for rinse-off use.

30. The conditioning composition of claim 1 which provides silicone deposition of about 50 ppm or more after rinsed off from the hair.

31. The conditioning composition of claim 30 which provides silicone deposition of about 100 ppm or more after rinsed off from the hair.

32. The conditioning composition of claim 31 which provides silicone deposition of about 200 ppm or more after rinsed off from the hair.

33. The conditioning composition of claim 32 which provides silicone deposition of about 400 ppm or more after rinsed off from the hair.

34. A method of conditioning hair, the method comprising following steps:
(i) after shampooing hair, applying to the hair an effective amount of the conditioning composition of claim 1 for conditioning the hair; and
(ii) then rinsing the hair.
